(19) United States
(12) Patent Application Publication
(10) Pub. No.: US 2010/0140241 A1
(43) Pub. Date: Jun. 10, 2010

(54) Auto-darkening viewing modules and control modules for welding light protection

(75) Inventors: Edward L. Martin, Sharon, MA (US); James P. Watkins, Taunton, MA (US); Jason Hsieh (Chien-Hsing), Taoyuan City (TW)

Correspondence Address: PEARSON & PEARSON, LLP, 10 GEORGE STREET, LOWELL, MA, 01852, US

(73) Assignee: A.C.E. INTERNATIONAL COMPANY, INC., TAUNTON, MA (US)

(21) Appl. No.: 12/589,029

(22) Filed: Oct. 16, 2009

Related U.S. Application Data
(62) Division of application No. 11/516,817, filed on Sep. 7, 2006.
(60) Provisional application No. 60/715,879, filed on Sep. 9, 2005.

(51) Int. Cl. B23K 9/10; A61F 9/06

(52) U.S. Cl. 219/130.21; 2/8.8

(57) ABSTRACT

A welding machine having a first receiver for remote control of the operating parameters, a viewing module having a second receiver for remotely controlling the operating parameters of the viewing module, and a control module for controlling the operating parameters of the welding machine and the viewing module wherein the control modules and the viewing modules may be mounted in a helmet. A master control module is provided for remotely controlling a plurality of control modules wherein each control module controls a viewing module. Each pair of control module and viewing ,module controlled by the master control module may be mounted in a helmet.

### CROSS-REFERENCE TO RELATED APPLICATIONS

This application claims the benefit of Provisional Application No. 60/715,879, filed Sep. 9, 2005 which is incorporated herein by reference.

### BACKGROUND OF THE INVENTION

1. Field of the Invention

This invention relates generally to the modular design of an auto-darkening welding filter of a welder's helmet, and more particularly to the mechanical packaging and the signaling of a control module, that controls the auto-darkening filter viewing module comprising a glass slide, polarizers, liquid crystal display (LCD), and UV/IR filter, and provides power for the control module, which is packaged separate from the viewing module.

2. Description of Related Art

Auto-darkening welding filters in helmets are widely used in the welding and cutting torch technology in order to protect the eyes of workers. Welding filters typically comprise a liquid crystal display (LCD) having control electronics and power circuits included within the filter which results in a reduced amount of viewing area for the worker wearing the helmet. In addition, considerable parts are required resulting in added costs for multiple parts procurement and inventory control.

U.S. Pat. No. 5,302,815, issued Apr. 12, 1994 to André M. Eggenschwiler and assigned to Optrel AG of Switzerland discloses a light protection apparatus for a welder's helmet comprising an electrically controllable light protection filter element, an optic-electric transducer element, a bridge member, two sensor elements, a real light protection filter element, a light protection cassette and control means including light sensitive sensors connected to the light protection filter element to control the optical transmission of the light protection filter element. However, the potential viewing area not only comprises an electrically controllable filter, but also an opto-electric transducer element, a bridge and two sensors which are all within the same package.

U.S. Pat. No. 5,533,206 issued Jul. 9, 1996 to Michael J. Petrie et al. and assigned to Jackson Products, Inc. of Belmont, Mich. describes a welding helmet with removable electronic quick change cartridge comprising an LCD lens, solar cells, photo sensor cells, and a circuit board. The photo sensor cells are on opposite sides of the circuit board within the cartridge. Having a circuit board within the cartridge reduces the space available for the LCD lens within an EQC cartridge.

U.S. Pat. No. 5,959,705, issued Sep. 28, 1999 to John D. Fergason and assigned to OSD Envizion, Inc. discloses a welding helmet having a welding lens with an integrated display and switching system. The switching system, including a deflectable cover plate, switch electrodes and a support, is associated with an automatic light shutter. The shutter includes liquid crystals which are operated by circuitry. The support may be a circuit board such as a rigid or flexible printed circuit board. The circuit board is retained within the housing. The circuitry is attached to the shutter and display and all are assembled in the housing of the welding lens cartridge assembly. Hence, all the components for the control, power, and welding lens with integrated display and switching are all located within the same package.

U.S. Pat. No. 6,070,264, issued Jun. 6, 2000 to Thomas J. Hamilton et al., and assigned to Jackson Products, Inc. of Chesterfield, Mo. discloses a welding helmet having a shutter assembly with auto-darkening and manually adjustable lens shade control. The electronic controls are within the shutter assembly, and the housing allows for two PC boards to be mounted above and below an optical shutter with a flexible cable interconnecting the two PC boards so as to not interfere with or obscure a user's vision. However, all the electronics are located within the shutter assembly and reduced space is available for viewing via the optical shutter.

U.S. Pat. No. 6,552,316, issued Apr. 22, 2003 to Young Dawn Bae and assigned to Otus Co., Ltd. discloses a helmet with a glare protecting device having an intensity control switch disposed on the exterior of the protective mask or helmet. The glare protecting device has a controller to regulate a glare protecting plate to protect a worker's eyes from a high intensity light. The controller is a microcomputer and controls ON/OFF of the glare protecting plate in the glare protecting device. However, the solar cell and the control circuitry are all located on or around the glare protecting device.

U.S. Pat. No. 6,557,174, issued May 6, 2003 to Edward L. Martin et al. and assigned to Optical Engineering Company, LLC of Taunton, Mass. discloses a replaceable self-contained expanded viewing light shield cartridge for a welding helmet comprising an optical mask, an LCD assembly, an opaque base, a second optical mask, an LCD driver, and printed circuit board 41. However, the control electronics are located within the light shield cartridge.

U.S. Pat. No. 6,796,652, issued Sep. 28, 2004 to Rico Sonderegger and assigned to Optrel AG of Switzerland discloses a glare-protection device with a screened evaluation circuit (FIG. 3). An electronic circuit is attached to the internal surface of a printed circuit board. The electronic circuit comprises the evaluation circuit and a driving circuit. However, the electronic circuits are located within the glare-protecting device.

### SUMMARY OF THE INVENTION

Accordingly, it is therefore an object of this invention to provide a welding helmet having an auto darkening filter comprising a viewing module and a control module packaged separate from the auto-darkening viewing module.

It is another object of this invention to provide a welding helmet having an auto-darkening viewing module with an electronic control module packaged separate from the auto-darkening viewing module and having control knob shafts protruding through the welding helmet for ease of adjustment of the filter by a user.

These and other objects are further accomplished by a welding helmet comprising a housing, an auto-darkening viewing module mounted in a front face of the housing, and a remote control module mounted in the welding helmet, separate from the viewing module, for controlling the viewing module. The control module comprises at least one variable control shaft extending through the housing or other means of control for a user to adjust the auto darkening viewing module. The control module comprises means for controlling functions in the control module, the controlling functions means being provided on the surface of the control module. The helmet comprises means for providing signals between the control module and the auto-darkening viewing module. The means for providing electrical signals between the control module and the auto-darkening viewing module comprises a wired cable. The means for providing signals between the control module and the auto-darkening viewing module comprises wireless apparatus.

The objects are further accomplished by providing a Welding Apparatus comprising a welding machine, the welding machine comprises a first receiver for remote controlling the operating parameters of the welding machine, a viewing module, the viewing module comprises a second receiver for remote controlling the operating parameters of the viewing module, and a control module for controlling the operating parameters of the welding machine and the viewing module.

The objects are further accomplished by providing a control system for a plurality of welding helmets comprising a master control module for remote controlling a plurality of control modules, and a plurality of viewing modules, each of the viewing modules being controlled by at least one of the plurality of control modules.

The objects are further accomplished by providing a control module comprising means for providing a predetermined voltage for a viewing module, means attached to the viewing module for sensing a welding light in front of the viewing module, means, coupled to the light sensing means, for controlling a dark mode and a light mode of the viewing module; and means coupled to the controlling means for quickly initiating the dark mode of the viewing module. The control module comprises means for detecting when a VCC voltage is below a minimum predetermined value. The viewing module comprises a liquid crystal display. The control module comprises means for delaying the viewing module from coming out of the dark mode. The control module comprises means coupled to a user-controlled variable signal for generating a shade signal which causes the viewing module to be adjusted between the light mode and the dark mode.

The objects are further accomplished by a method of providing a welding helmet comprising the steps of providing a helmet housing, mounting an auto darkening viewing module in a front face of said housing, and mounting a control module in said welding helmet separate from said viewing module.

Additional objects, features and advantages of the invention will become apparent to those skilled in the art upon consideration of the following detailed description of the preferred embodiments exemplifying the best mode of carrying out the invention as presently perceived.

### BRIEF DESCRIPTION OF THE DRAWINGS

The appended claims particularly point out and distinctly claim the subject matter of this invention. The various objects, advantages and novel features of this invention will be more fully apparent from a reading of the following detailed description in conjunction with the accompanying drawings in which like reference numerals refer to like parts, and in which:

FIG. 1 is a front perspective view of a welding helmet showing control knobs of an auto-darkening welding filter attached to variable resistor shafts extending through the housing.

FIG. 2 is a functional block diagram of a modular auto-darkening welding filter for a welding helmet according to the present invention;

FIG. 3 is an inside rear view of the welding helmet of FIG. 1 showing an auto-darkening filter having a separate control module with control shafts protruding through the side of the welding helmet, as well as other user controls on the control module, and the control module being attached to the helmet adjacent to the viewing module; and

FIG. 4 is an inside rear view of a welding helmet showing an alternate embodiment of an auto-darkening filter having a separate control module attached to the welding helmet adjacent to and below the viewing module.

FIG. 5 is a block design of a welding system including a welding machine, a viewing module of a welding helmet and a control module.

FIG. 6 is a block diagram of a control system having a master control module for setting the parameters of a plurality of control modules each of which determines the behavior of a viewing module.

FIGS. 7A, 7B and 7C combined are a schematic diagram of a control module.

### DESCRIPTION OF ILLUSTRATIVE EMBODIMENT

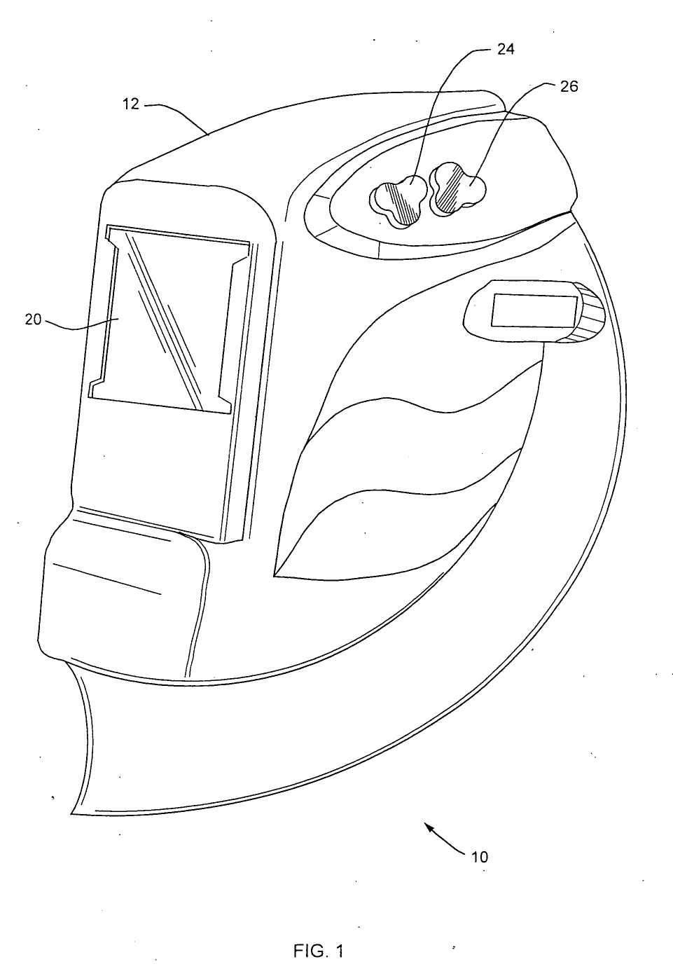

Referring to FIG. 1, FIG. 1 is a front perspective view of a welding helmet 10 having an auto-darkening welding filter with control knobs 24, 26 attached to variable resistor shafts 25, 27 extending through the housing 12 from a control module 18 located inside the welding helmet 10 for a user to adjust the auto-darkening full view welding filter in accordance with the present invention.

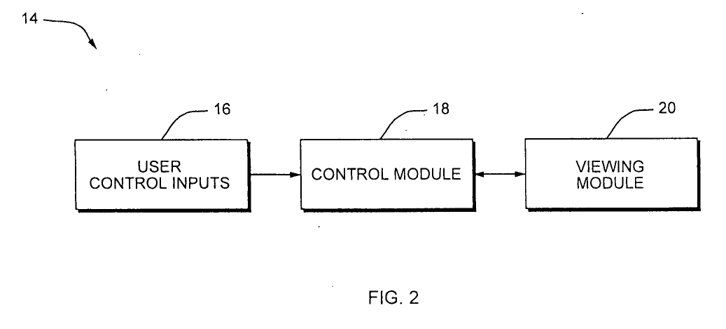

Referring to FIG. 2, FIG. 2 is a functional block diagram showing a modular auto-darkening welding filter system 14 for the welding helmet 10 comprising a viewing module 20 controlled by a control module 18 physically separated from the viewing module 20 and control inputs 16 provided by a user to the control module 18. The parameters of the viewing module 20 that are controlled by the control module 18 include shade, sensitivity, and delay. Communication between the control module 18 and the viewing module 20 may be accomplished using a mechanical cable and connectors, a magnetic signal, an electromagnetic signal or combinations thereof. The welding helmet 10 may be embodiment by model Cobra-911 which includes the auto-darkening welding filter comprising the viewing module 20 and the control module 18 which may be embodied by Model 6000VI4 manufactured by ArcOne, a Division of A.C.E. International Company, Inc. of Taunton, Mass.

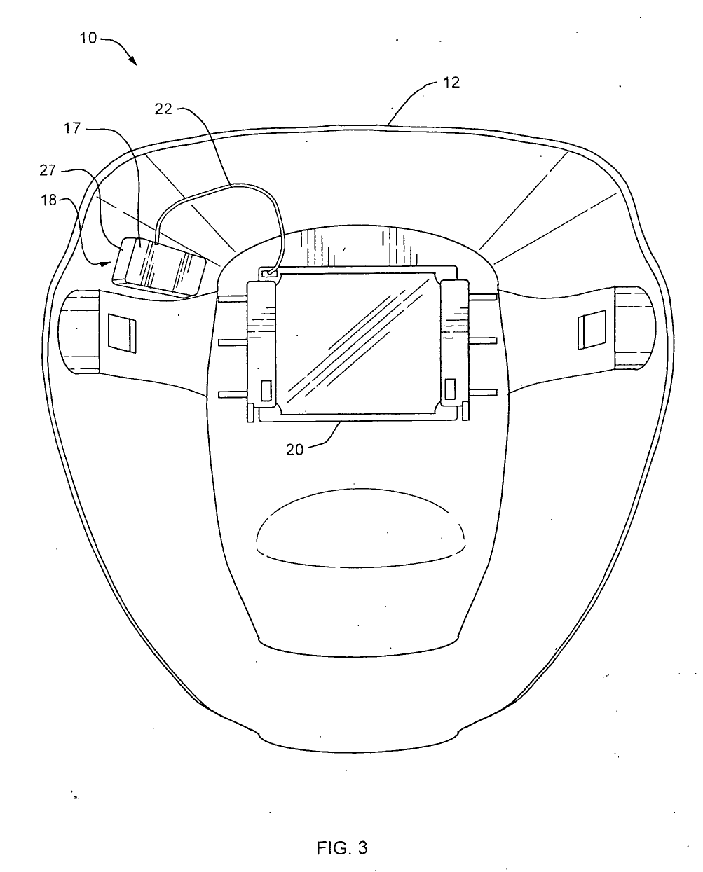

Referring to FIG. 3, FIG. 3 is an inside rear view of a welding helmet 10 showing an auto-darkening welding filter having the viewing module 20 and the control module 18 which is mounted on the side of the housing 12 of the welding helmet 10 separate from the viewing module 20. User control inputs 16 are provided to the control module 18 by turning the control knobs 24, 26 on variable resistor shafts 25, 27 extending from the control module 18 through the side of the housing 12 of the welding helmet 10 and having knobs 24, 26 (FIG. 1) attached to the end of the shafts for user adjustment. Other user control adjustments 17 are located on the case of control module 18. A battery holder 29 is attached to the side of the control module 18. An electrical cable 22 is connected between the filter 20 and the control module 18.

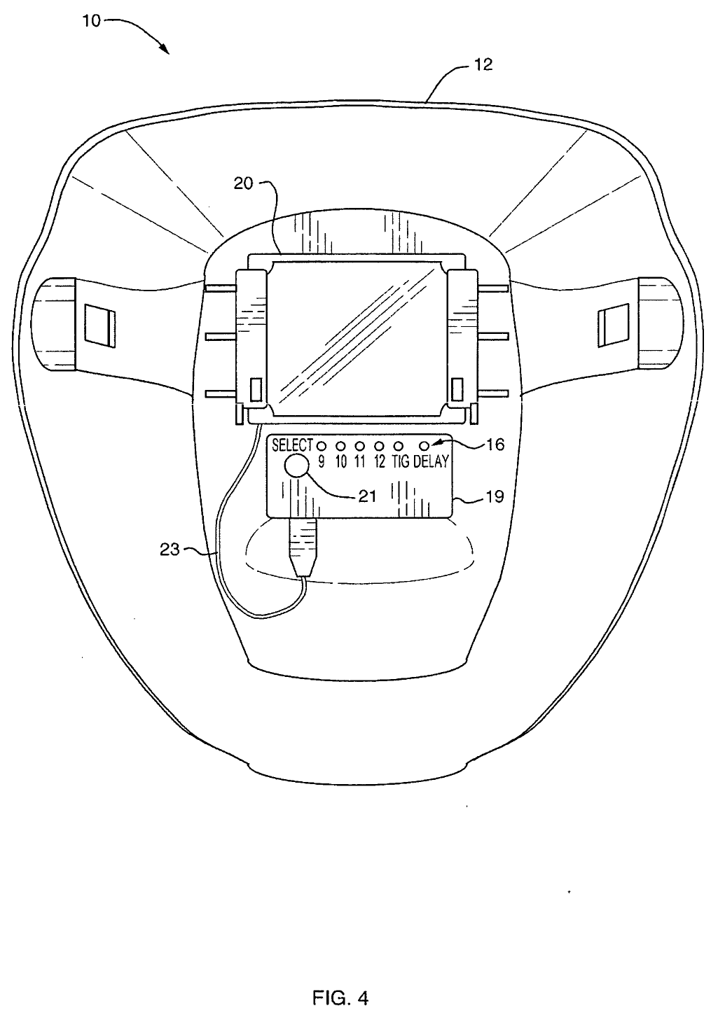

Referring to FIG. 4, FIG. 4 is an inside rear view of an alternate embodiment of the welding helmet 10 showing an auto-darkening filter comprising the viewing module 20 with the control module 19 mounted separate from and below the viewing module 20. An electrical cable 23 is connected between the viewing module 20 and the control module 19. In this embodiment of the control module 19, control switches 21 are provided on the outside surface of the module 19 for a user to provide control inputs 16 for adjusting the viewing module 20.

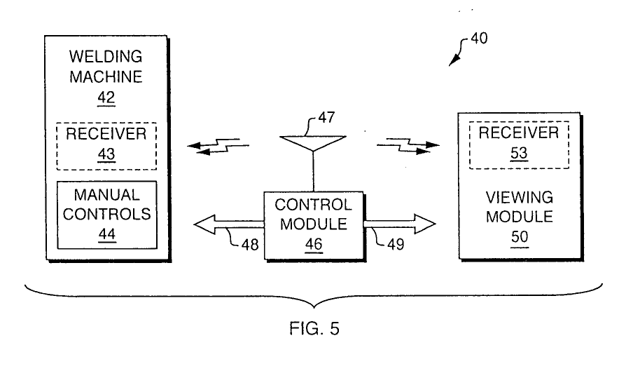

Referring to FIG. 5, FIG. 5 is a block diagram of a welding system 40 comprising a welding machine 42, a control module 46 and a viewing module 50. The welding machine comprises a receiver 43 for receiving control signals from a control module 46 which is wireless having an antenna 47 or it may be connected to the welding machine 42 or the viewing module 50 via cables 48, 49. The control module 46 controls the operating characteristics of the welding machine 42, and it also controls the viewing module 50 via receiver 53 both of which are typically mounted in a welding helmet and controlled by an operator.

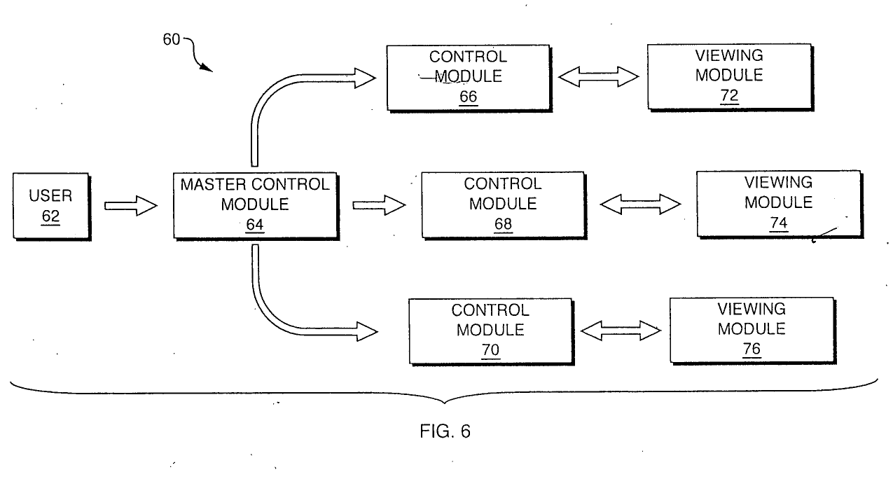

Referring to FIG. 6, FIG. 6 is a block diagram of a control system 60 comprising a master control module 64 which receives inputs from a user 62 for setting the parameters of a plurality of control modules 66, 68, 70, each of which determines the behavior of a viewing module 72, 74, 76. Each viewing module 72, 74, 76 is typically mounted in a welding helmet, so that the user can look through the viewing module 72, 74, 76 to observe a welding operation. The master control module 64 oversees multiple control modules 66, 68, 70 to coordinate the behavior of the viewing modules 72, 74, 76. The communications between the master control module 64 and the control modules 66, 68, 70 is wireless, and the communication between the control modules 66, 68, 70 and the viewing modules 72, 74, 76 is wireless and/or wired.

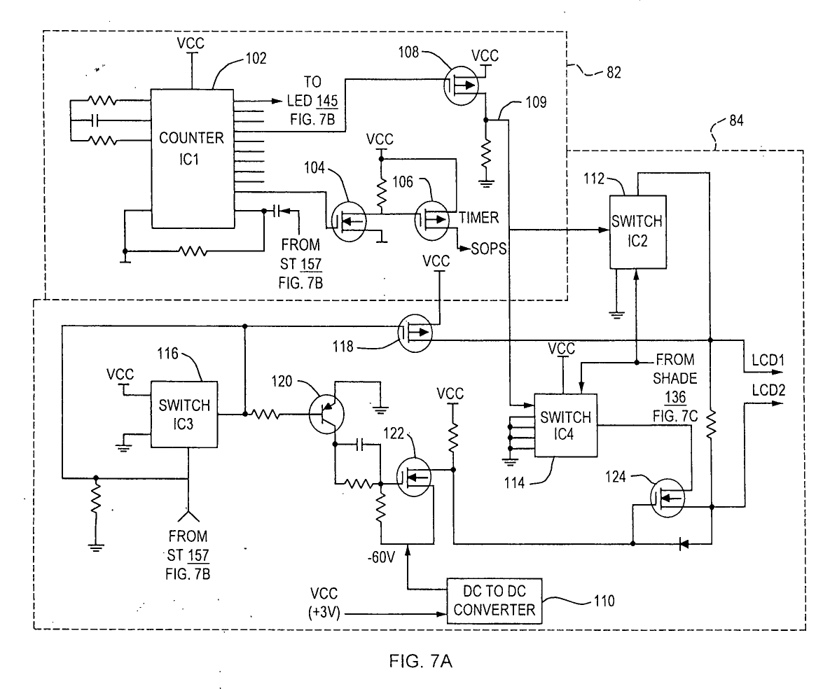

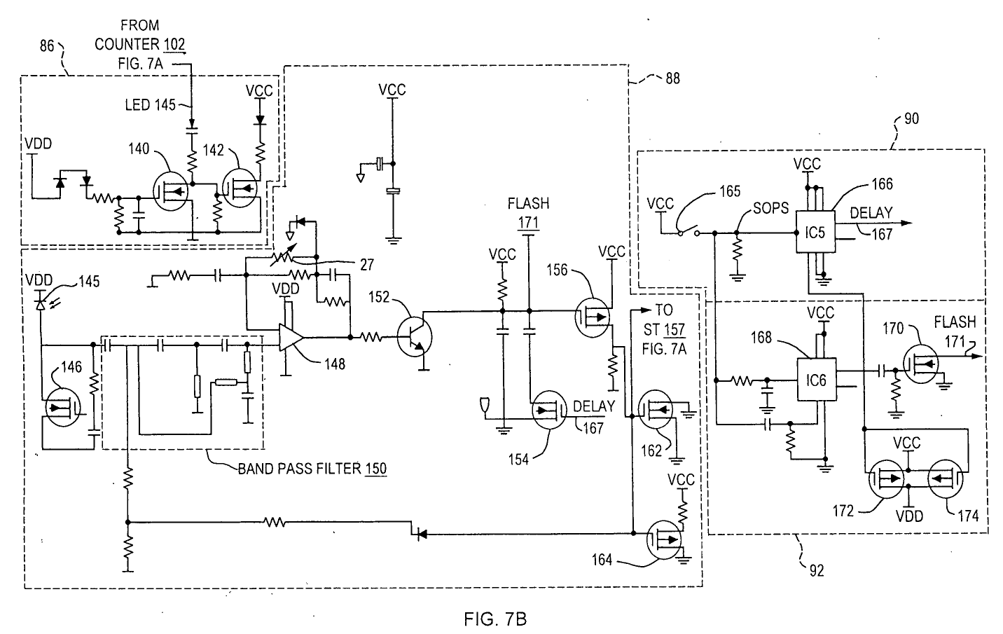

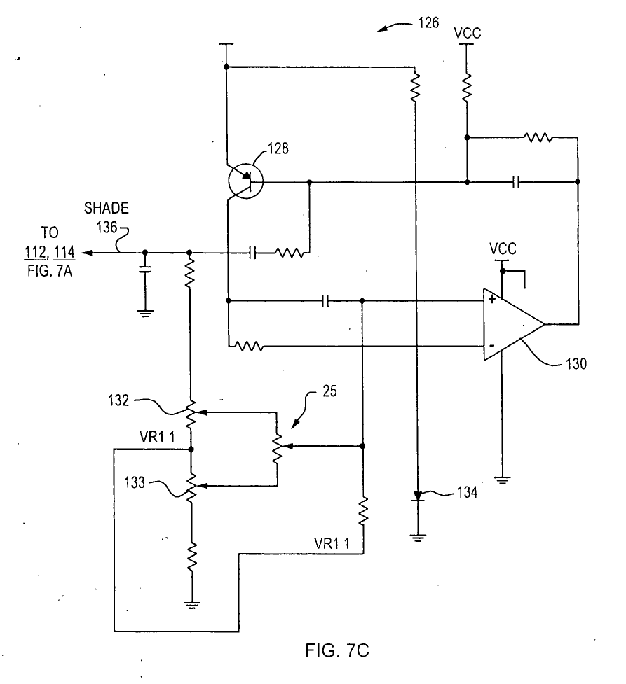

Referring to FIGS. 7A, 7B and 7C, a schematic diagram is shown of the circuitry in the control module 18 for controlling the viewing module 20 and for controlling welding apparatus such as a welding machine 42 or a viewing module 50 in a welding helmet.

The control module 18 comprises an Auto Power Off Circuit 82, a Viewing Module Control Circuit 84, a Low Power Detect Circuit 86, a Sensor Circuit 88, a Delay Circuit 90, a Feedback Flash Circuit 92, a Shade Voltage Generator 126, and a DC to DC Converter 110.

Referring to FIG. 7A, the auto power off circuit 82 is used to turn-off the power to the circuits in the control module 18 (or 19,46, 66, 68, 70) and likewise the power to the corresponding viewing modules 20 (or 50, 72, 74, 76). A counter 102 is designed to time-out every 15 minutes. An ST signal 157 from the Sensor Circuit 88 (FIG. 7B) provides a reset signal to the counter 102. Outputs from the counter 102 are coupled to Mosfet 104 and Mosfet 108. Mosfet 104 is connected to Mosfet 106 and generates a Shut-Off Power Source (SOPS) signal which connects to a delay circuit 90 in FIG. 7B. The output 109 of Mosfet 108 is connected to switches 112 and 114 in the Viewing Module Control Circuit 84. In the circuits of FIG. 7A, VCC equals 3 VDC.

The Viewing Module Control Circuit 84 comprises three integrated circuit switches 112, 114, 116, a bipolar transistor 120 and Mosfets 118 and 122. The switch 116 receives a single turn-on (ST) signal 157 from the Sensor Circuit 88 in FIG. 7B which enables transistor 120 and Mosfet 122 to transfer a −60V input to output LCD2. A DC to DC converter 110 receives Vcc (+3V) and converts it into a −60V output. Such a converter 110 is known to one of ordinary skill in the art. LCD1 and LCD2 outputs connect to the Viewing Module 20 and provide an initial −60V pulse to quickly switch the viewing module 20 from a light mode to a dark mode, and the Viewing Module 20 is then maintained in the dark mode by switches 112 and 114 which provide a switching signal from +3 volts to −3 volts to maintain the Viewing Module 20 in the dark mode. The switches 112 and 114 are enabled by signal 109 from the Auto Power Off Circuit 82 and they convert a SHADE Signal 136 from a SHADE Voltage Generator 126 (FIG. 7C) to AC signals LCD1 and LCD2 for driving the viewing module 20. The counter 102 may be embodied by part no. 5IST4047.

In FIG. 7A, the Mosfet 104 may be embodied by part no. 5QRK2N7002, Mosfet 122 and 124 may be embodied by part no. 5QBSBST82, Mosfets 106, 108 and 118 may be embodied by part no. 5QBSBSS84, IC switches 112 and 114 may be embodied by part no. 5QRK2N7002, transistor 120 may be embodied by part no. 5QBC857C, all manufactured by Phillips Electronics of Taiwan.

Referring now to FIG. 7C, the Shade Voltage Generator 126 comprises variable resistors 132, 133 and 25. A Control Knob 24 of variable resistor 25 is shown in FIG. 1 which is adjusted by a user to adjust the auto-darkening viewing module 20. A signal from variable resistor 25 is fed to an amplifier/comparator 130, the output of which is coupled to a transistor 128 which provides the SHADE signal 136. Variable resistor 132 acts as a high level limiter and variable resistor 133 acts as a low level limiter. An LED 134 provides a 1.4V reference to an input of the amplifier/comparator circuit 130.

In FIG. 7C, transistor 128 may be embodied by part no. 5QBC857C, amplifier/comparator 130 may be embodied by part no. 5IS0TLC271 manufactured by Phillips Electronics of Taiwan, and LED diode 134 may be embodied by part no. LTST-C190KRKT, manufactured by Lite-On Technology Corp.

Referring now to FIG. 7B, the Low Power Detect Circuit 86 comprises Mosfets 140 and 142, and it monitors VDD (+3V) and VCC (+3V). When a low voltage is detected such as below 2.7V Mosfet 140 switches causing a light emitting diode (LED) 144 to turn-on. The LED 144 flashes indicating a “low voltage” condition in response to the LED signal from counter 102 (FIG. 7A). The MOSFETS 140 and 142 may be embodied by part no. 5QBSNSS84 and part no. 5QRK2N7002 all manufactured by Phillips Electronics of Taiwan.

The Sensor Circuit 88 comprises a Mosfet 146 connected to a photoelectric diode 145 which is coupled to a band pass filter 150 circuit for sensing a welding light frequency. The photoelectric diode 145 is actually located on the outside edge of the viewing module 20 (FIG. 1). An operational amplifier/comparator 148 receives the band pass filter 150 output, and bipolar transistor 152 amplifies the detected welding light signal frequency. The variable resistor 27 is adjusted by a user by moving knob 26 to control the sensitivity of the amplifier/comparator 148 by adjusting the threshold at which the amplifier/comparator reacts to make the viewing module 20 go dark. The output of transistor amplifier 152 is coupled to Mosfet 156 which generates an single turn-on (ST) signal 157 and sends it to the Viewing Module Control Circuit 84 (FIG. 7A). The single turn-on ST signal 157 will quickly initiate the darkening of the Viewing Module 20 in approximately 0.1 milliseconds.

The Delay Circuit 90 operates in conjunction with switch 165 and the Feedback Flash Circuit 92. When the switch 165 is closed momentarily, power is initiated to the control module 18, and if the switch 165 is closed for a few seconds, a DELAY signal 167 is generated. The Delay Circuit 90 comprises a flip-flop IC circuit 166 which generates the Delay signal 167 which is normally approximately 100 milliseconds and is coupled to Mosfet 154 in the Sensor Circuit 88. The delay produced is to delay the Viewing Module 20 from coming out of the dark mode. When switch 165 is closed by a user, the delay becomes approximately 2.0 seconds.

The Feedback Flash Circuit 92 comprises a flip-flop circuit 168 which receives a voltage signal when the switch 165 is closed setting the flip-flop IC circuit 168, and it drives Mosfet 170 and generates a FLASH signal 171 which is coupled to Mosfet 156 and causes the ST signal to be generated which causes the Viewing Module 18 to go “dark”. The Feedback Flash circuit 92 comprises Mosfets 172 and 174 which are controlled by the output of flip-flop IC circuit 168 for controlling the power of the viewing module 20 so it stays in the light (???) mode.

In FIG. 7B, Mosfets 146, 154, 162 and 170 may be, embodied by part no. 5Q5K2N7002, Mosfets 156, 164, 172 and 174 may be embodied by part no. 5QBSBSS84, amplifier/comparator may be embodied by part no. 5QBC847C, transistor 152 may be embodied by part no. 5IS0TLC271, and IC 166 and IC 168 may be embodied by part

1-6. (canceled)

7. In combination:
a welding machine, said welding machine comprises a first receiver for remote controlling the operating parameters of said welding machine;
a viewing module, said viewing module comprises a second receiver for remote controlling the operating parameters of said viewing module; and
a control module for controlling the operating parameters of said welding machine and said viewing module.

8. In combination:
a master control module for remotely controlling a plurality of control modules; and
a plurality of viewing modules, each of said viewing modules being controlled by at least one of said plurality of control modules.

9-21. (canceled)

22. The combination as recited in claim 7 wherein said welding machine comprises manual controls for controlling operating parameters of said welding machine in addition to said first receiver for receiving remote control signals.

23. The combination as recited in claim 7 wherein said combination comprises a helmet having said receiving module and said control module mounted therein for operation of said control module by a wearer of said helmet.

24. The combination as recited in claim 23 wherein said wearer of said helmet controls the operating parameters of said welding machine remotely at said helmet.

25. The combination as recited in claim 8 wherein each of said viewing modules is mounted in a helmet for protection of a wearer of said helmet observing a welding operation.

26. The combination as recited in claim 8 wherein communications between said master control module and each of said control modules comprises wireless communication.

27. The combination as recited in claim 8 wherein communications between each of said viewing modules and each of said associated control modules comprise one of a group of wireless and wired communications.

28. The combination as recited in claim 8 wherein each of said control modules comprises:
an auto power off circuit for shutting off power to said control module under predetermined conditions;
a control circuit for said viewing modules;
a low power detached circuit;
a sensor circuit for sensing a welding light frequency;
a delay circuit for delaying said viewing module from coming out of a dark mode;
a feedback flash circuit;
a shade voltage generator; and
a DC to DC, converter for converting +3V to −60V.
